(19) United States
(12) Patent Application Publication
(10) Pub. No.: US 2003/0149447 A1
(43) Pub. Date: Aug. 7, 2003

(54) Barbed surgical suture

(76) Inventors: Steven David Morency, Sunnyvale, CA (US); Jeffrey S. Jones, Hillsborough, CA (US)

Correspondence Address: Gregory Smith & Associates, Suite 317, 3900 Newpark Mall Road, Newark, CA, 94560, US

(21) Appl. No.: 10/322,079

(22) Filed: Dec. 17, 2002

Related U.S. Application Data
(60) Provisional application No. 60/353,329, filed on Feb. 1, 2002.

(51) Int. Cl. A61L 17/00

(52) U.S. Cl. 606/228

(57) ABSTRACT

A barbed surgical suture is configured to grip the tissue through which it is inserted. The suture has a generally flat and elongated suture body with a multiplicity of barbs located along one or both of the lateral edges. The barbs are of sufficient size and appropriate geometry for fastening the tissue and achieving closure of an incision or wound without the need for tying knots in the suture. The barbed surgical suture may be configured as a single-ended suture with all of the barbs aligned to allow the suture to move through tissue in one direction only. Alternately, it may be configured as a double-ended suture with the barbs on a first end portion aligned to allow the suture to move through tissue in a one direction and the barbs on a second end portion aligned to allow the suture to move through tissue in the opposite direction. The suture needles may be permanently attached or removably attached to the barbed surgical suture or, alternatively, the suture needle may be integrally formed with the barbed surgical suture. Methods are described for manufacturing the barbed surgical suture by stamping, cutting or progressive die cutting the suture out of flat material, or by chemically etching the suture out of flat material and or by injection molding. Optionally, specialized coatings may be added to enhance the performance of the barbed surgical suture.

### CROSS REFERENCE TO OTHER APPLICATIONS

This application claims the benefit of U.S. Provisional Patent Application Ser. No. 60/353,329, filed Feb. 1, 2002, the specification of which is hereby incorporated by reference.

### FIELD OF THE INVENTION

The present invention relates generally to surgical sutures. More particularly, it relates to a surgical suture with barbs that grip the tissue and eliminate the need for tying knots in the suture.

### BACKGROUND OF THE INVENTION

Proper healing of surgical incisions and wounds often requires closure of the incision or wound with a surgical fastener. Sutures have long been the gold standard in surgical fasteners because of their versatility and reliability. In applications where cosmesis is important, sutures can be used to achieve a highly cosmetic closure. However, suturing is a time consuming process. Therefore, in recent years, surgical staples have replaced sutures in many internal and external closure applications because of their speed and convenience. However, the cosmesis of closures made with surgical staples is seldom as good as a sutured closure. What would be highly desirable and heretofore unavailable is a surgical fastener that provides the speed and convenience of surgical staples with the potential for good cosmesis that can be achieved with sutures.

Barbed sutures offer the potential of rapid, reliable closure with good cosmesis. However, previous attempts to make a barbed suture have not met with commercial or clinical success. In large part this is due to the difficulty of manufacturing a barbed suture and the compromises that must be made in the configuration of the barbed suture to accommodate known manufacturing methods.

Alcamo, U.S. Pat. No. 3,123,077, describes a Surgical Suture with a roughened surface or barbs to prevent slippage of the suture after each stitch. The surgical suture is intended for use with a Surgeon's Suturing Device, such as described in U.S. Pat. No. 2,988,028, which creates a running or looped stitch for closing an incision in a patient. This patent does not describe any method for manufacturing the surgical suture and the configuration of the surgical suture disclosed would not lend itself to standard manufacturing processes.

Buncke, U.S. Pat. No. 5,931,855, describes a Surgical Method Using One-Way Suture wherein single-ended barbed sutures are placed through both sides of a surgical incision, then the trailing ends of the sutures from the two sides are bonded together to close the incision. This patent describes a method for manufacturing the barbed surgical suture by cutting the barbs into a suture body using a mechanical cutting blade or a laser. This method has not proven to be reliable or economical enough for commercial production of barbed surgical sutures. Furthermore, barbed surgical sutures made by this method are necessarily limited in the size and geometry of the barbs that can be produced.

What is desirable, therefore, is to provide a barbed suture that overcomes these deficiencies in the prior art and, in particular, to provide a barbed suture that is readily manufacturable with barb features of sufficient size and appropriate geometry for fastening surgical closures without the need for knots or complicated stitching to achieve closure.

### SUMMARY OF THE INVENTION

In keeping with the foregoing discussion, the present invention takes the form of a surgical suture with barbs configured to grip the tissue through which it is inserted. The suture has a generally flat and elongated suture body with a multiplicity of barbs located along the lateral edges of the elongated suture body. The barbs are of sufficient size and appropriate geometry for fastening the tissue and achieving closure of an incision or wound without the need for tying knots in the suture. This feature allows the barbed surgical suture to achieve excellent closure results, superior even to standard knotted sutures. When appropriate, the barbed surgical suture may also be used to achieve superior cosmetic results.

The barbed surgical suture may be configured as a single-ended suture with all of the barbs aligned to allow the suture to move through tissue in one direction and to resist moving through the tissue in the other direction. The single-ended barbed surgical suture may be manufactured with a suture needle attached to the leading end of the suture and optionally may include a T-shaped member or other stop device on the trailing end of the suture. The suture needle may be permanently attached or removably attached to the barbed surgical suture or, alternatively, the suture needle may be integrally formed with the barbed surgical suture.

The barbed surgical suture may also be configured as a double-ended suture with the barbs on a first end portion aligned to allow the suture to move through tissue in a one direction and the barbs on a second end portion aligned to allow the suture to move through tissue in the opposite direction. The double-ended barbed surgical suture may be manufactured with suture needles attached at one or both ends of the suture. The suture needles may be permanently attached or removably attached to the barbed surgical suture or, alternatively, the suture needles may be integrally formed with the barbed surgical suture.

The configuration of the barbed surgical suture lends itself to economical methods of manufacture. Methods are described for manufacturing the barbed surgical suture by stamping or cutting the suture out of flat material, by chemically etching the suture out of flat material and by injection molding. Optionally, specialized coatings may be added to enhance the performance of the barbed surgical suture. The coatings may be used to improve lubricity, reduce inflammatory response, aid insertion, enhance healing, etc. Examples of coatings that may be beneficial include silicone, TEFLON, metal plating, pharmaceutical materials, etc. dr

### BRIEF DESCRIPTION OF THE DRAWINGS

FIG. 1A shows a single-ended barbed surgical suture constructed according to the present invention.

FIG. 1B shows a cross section of a first configuration of the barbed surgical suture of FIG. 1A.

FIG. 1C shows a cross section of an alternate configuration of the barbed surgical suture of FIG. 1A.

FIG. 1D is an enlarged fragmentary view of a portion of the barbed surgical suture of FIG. 1A.

FIG. 1E shows a cross section of another alternate configuration of the barbed surgical suture having tapered barbs.

FIG. 1F shows an enlarged fragmentary view of a portion of an alternate barbed surgical suture having curved barbs.

FIG. 1G shows an enlarged fragmentary view of a portion of another alternate barbed surgical suture having rounded barbs.

FIG. 1H shows an alternate single-ended barbed surgical suture constructed with a straight needle.

FIG. 1I shows an alternate single-ended barbed surgical suture constructed with a broadened distal tip.

FIG. 2 shows the single-ended barbed surgical suture of FIG. 1A being used to close a surgical incision.

FIG. 3 shows the completed closure of the surgical incision using the single-ended barbed surgical suture of FIG. 1A.

FIG. 4 shows a double-ended barbed surgical suture constructed according to the present invention.

FIG. 5 shows the double-ended barbed surgical suture of FIG. 4 being used to close a surgical incision.

FIG. 6 shows the completed closure of the surgical incision using the double-ended barbed surgical suture of FIG. 4.

FIG. 7A shows a single-ended barbed surgical suture with an alternate barb configuration.

FIG. 7B is an enlarged fragmentary view of a portion of the barbed surgical suture of FIG. 7A.

FIG. 8A shows a single-ended barbed surgical suture with another alternate barb configuration.

FIG. 8B is an enlarged fragmentary view of a portion of the barbed surgical suture of FIG. 8A.

FIGS. 9-10 illustrate two steps of a manufacturing process for producing a barbed surgical suture with an integrally formed suture needle.

FIG. 11 illustrates a manufacturing process for producing a barbed surgical suture from flat material.

FIGS. 12A and 12B illustrates an elongated wound closed with a barbed surgical suture.

### DETAILED DESCRIPTION OF THE INVENTION

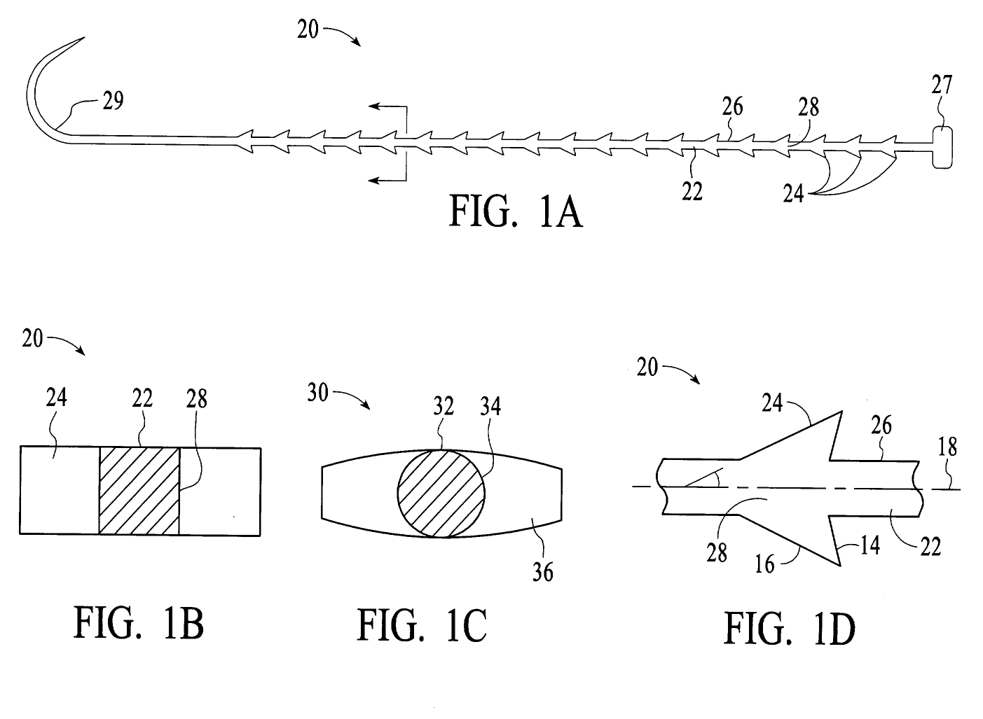

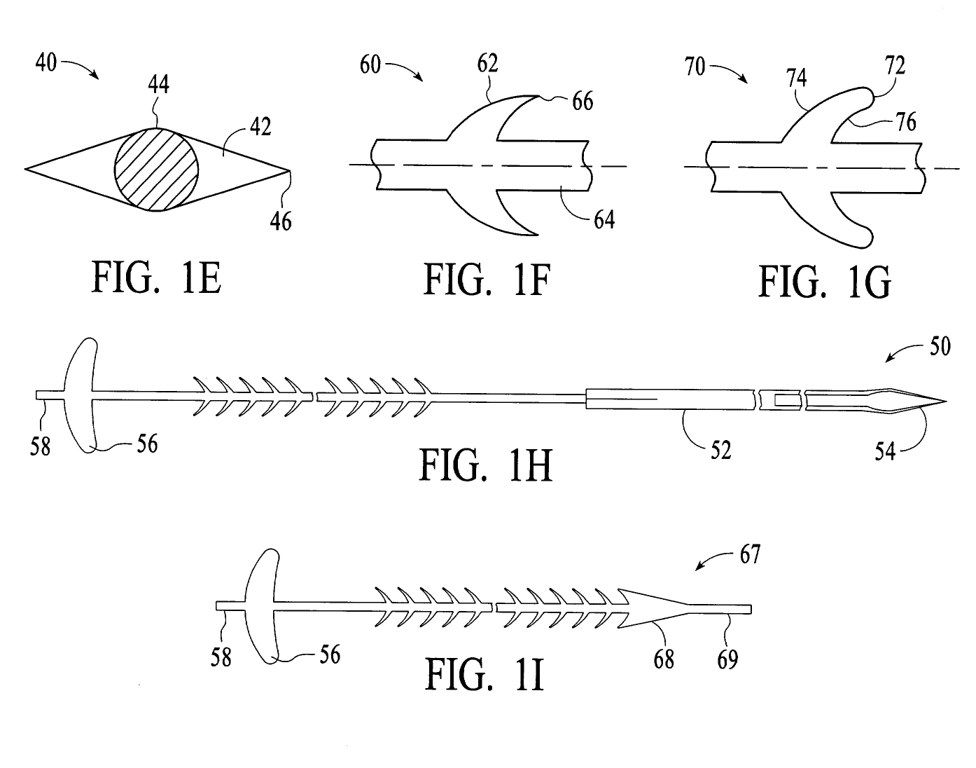

FIG. 1A shows a barbed surgical suture 20 constructed according to the present invention. The suture 20 has a generally flat and elongated suture body 22 with a multiplicity of barbs 24 located along the lateral edges 26 of the elongated suture body 22. The barbs 24 may be arranged in opposing pairs, as shown, or alternatively, the barbs 24 may be arranged in a staggered pattern along the lateral edges 26 of the elongated suture body 22 or on a single side. The generally flat configuration of the elongated suture body 22 lends itself to a number of different manufacturing processes, as will be discussed in greater detail below. FIG. 1B shows a cross section of a first configuration of the barbed surgical suture 20 of FIG. 1A. In this configuration, the entire suture body 22 has a generally rectangular cross section, as do each of the barbs 24 and the connecting member 28 that extends between each pair of barbs 24. This flat configuration lends itself to manufacturing the barbed surgical suture 20 by forming the barbed surgical suture 20 from a flat stock material. This embodiment of the barbed surgical suture 20 has a curved suture needle 29.

FIG. 1C shows a cross section of an alternate configuration 30 of the barbed surgical suture of FIG. 1A. In this configuration, the rectangular cross section of the suture body 32 is modified by curving the upper and lower surfaces of the barbs 36 and giving the connecting member 34 that extends between each pair of barbs 36 a rounded cross section. This modified flat configuration lends itself to manufacturing the barbed surgical suture 30 by injection molding.

FIG. 1E shows a cross section of another alternate configuration 40 of the barbed surgical suture of FIG. 1A. This configuration has the barbs 42 tapered from the thickness of the suture body 44 to a point at the tip 46 of the barb 42.

FIG. 1H shows an alternate embodiment of the barbed surgical suture 50. This version of the barbed surgical suture 50 has a straight needle 52 with a beveled tip 54. The T-shaped end 56 is curved or rounded with a tab 58 extending outward from the end. The tab 58 provides an easy location to grip the end of the barbed surgical suture 50 with the fingers or forceps while the suture 50 is being manipulated. If desired, the tab 58 may be clipped off after the barbed surgical suture 50 is in place.

The barbs 24, 36, 42, 62, 72, 82, 92, 100, 140 are configured to allow the barbed surgical suture 20, 30, 40, 50, 60, 67, 70, 80, 90, 100 to move through tissue in one direction and to resist moving through the tissue in the other direction. The barbs are of sufficient size and appropriate geometry for fastening the tissue and achieving closure of an incision or wound without the need for tying knots in the suture. The barbs may be closely spaced along the suture body for situations where a high gripping force is needed or they may be spaced apart for situations where a less gripping force is needed. When the barbs are spaced apart, a narrow connecting member extends longitudinally between each of the barbs. The barbs may have a width from approximately 10% to approximately 40% of the width of the suture body and the connecting member may have a width from approximately 20% to approximately 80% of the width of the suture body. The barbed suture may be manufactured in almost any width and thickness, including widths and thicknesses corresponding approximately to the range of available diameters for standard sutures, which range from approximately 0.00004 to approximately 0.0530 inches. In one particularly preferred embodiment, the suture body is manufactured with a width in the range from approximately 0.0136 to approximately 0.0150 inches and a thickness in the range from approximately 0.0050 to approximately 0.0075 inches, corresponding approximately to a USP size for standard sutures in the range from approximately 4-0 to approximately 3-0. The barbed suture may be manufactured in a continuous length or may be manufactured in discrete lengths, for example in discrete lengths in the range from ½ inch to 2 feet or more.

The barbs 24, 36, 42, 62, 72, 82, 92, 100, 140 may be constructed in many different configurations. In an exemplary embodiment shown in FIG. 1A, the barbs have a width of approximately 32% of the width of the suture body. As shown in the enlarged fragmentary view in FIG. 1D, each barb is approximately triangular or wedge-shaped. The leading edge of each barb diverges from the centerline of the suture body at an acute angle, more preferably at an angle equal to or less than 45°, and most preferably at an angle between approximately 25° and approximately 30°. The trailing edge of each barb also diverges from the centerline of the suture body at an acute angle, more preferably at an angle between approximately 60° and approximately 78°, so that the trailing edge is slightly undercut. The acutely angled leading edge and the undercut trailing edge help to allow the barbs to flex inward so that the barbed surgical suture moves easily through the tissue in the forward direction, whereas when there is tension on the barbed surgical suture in the reverse direction the angled trailing edge helps to expand or flex the barbs inward or outward, biting into and grasping the tissue to resist rearward motion.

FIG. 1F shows an enlarged fragmentary view of a portion of an alternate barbed surgical suture 60. This embodiment has the barbs 62 curved back toward the body 64 of the suture 60. The curvature of the barbs 62 allows the barbed surgical suture 60 to more smoothly enter the tissue. However, when tension is placed on the barbed surgical suture 60 in the reverse direction, the tip 66 of the barb caused the barb 62 to straighten or even flex the barb 62 outward, thereby holding in the tissue to resist rearward motion of the barbed surgical suture 60.

FIG. 1G shows an enlarged fragmentary view of a portion of another alternate barbed surgical suture 70. In this embodiment, the barbs 72 are rounded on both the leading edge 74 and trailing edge 76. The rounded barbs 72 may be used in situations with delicate tissue, which is prone to tearing. The rounded barbs 72 allow the barbed surgical suture 70 to enter the tissue and hold with a minimal amount of tearing of the tissue.

FIG. 1I shows a side view of an alternate embodiment of the barbed surgical suture 67. In this embodiment, the distal end of the barbed surgical suture 67 has a narrow distal tip 69 and a broadened body portion 68 that form the needle section of the barbed surgical suture 67. The narrow tip 69 and broadened portion 68 assist with tissue penetration and handling of the barbed surgical suture 67.

In other embodiments, the barbs 24, 36, 42, 62, 72, 82, 92, 100, 140 may be different configurations along the length of the suture body 22, 32, 44, 64, 116. The changes in configuration may be in spacing or barb shape. For example, the barbs on the may be closely spaced and have pointed tips to provide maximum holding force. The center section may have more widely spaced barbs with rounded tip to minimize the trauma to the tissue. Alternate embodiments may use other combinations and configurations of the barbs for other purposes and specialty situations.

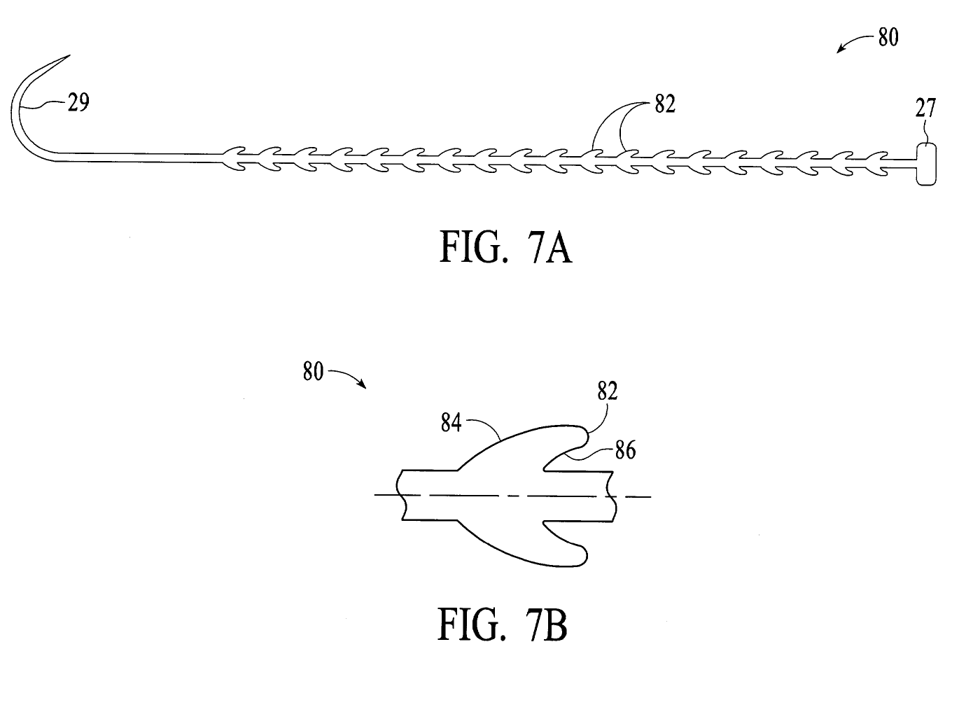

FIG. 7A shows a barbed surgical suture 80 with an alternate barb 82 configuration. FIG. 7B is an enlarged fragmentary view of a portion of the barbed surgical suture 80 of FIG. 7A. In this exemplary embodiment, the leading edges 84 of the barbs 82 have a convex curvature and the trailing edges 86 have a concave curvature.

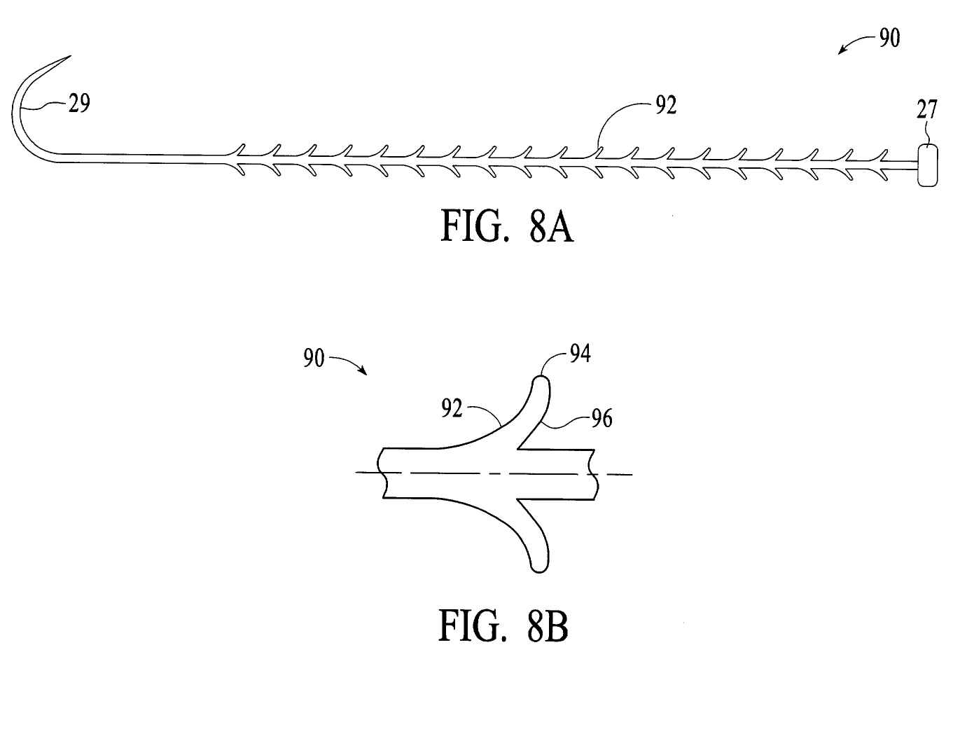

FIG. 8A shows a barbed surgical suture 90 with another alternate barb 92 configuration. FIG. 8B is an enlarged fragmentary view of a portion of the barbed surgical suture 90 of FIG. 8A. In this exemplary embodiment, the leading edges 94 of the barbs 92 have a concave curvature and the trailing edges 96 have a convex curvature.

The barbed surgical suture 20, 30, 40, 50, 60, 67, 70, 80, 90, 100 may be configured as a single-ended suture with all of the barbs 24, 36, 42, 62, 72, 82, 92, 100, 140 aligned to allow the suture to move through tissue in a forward direction and to resist moving through the tissue in the reverse direction. FIGS. 1A, 7A and 8A show examples of a single-ended barbed surgical suture constructed according to the present invention. The barbs on the single-ended barbed surgical suture may be made in many different configurations, including those shown in FIGS. 1D, 7B and 8B. The single-ended barbed surgical suture may be manufactured with either a curved or straight suture needle attached to the leading end of the suture. The suture needle may be permanently attached or removably attached to the barbed surgical suture or, alternatively, the suture needle may be integrally formed with the barbed surgical suture. Optionally, the single-ended barbed surgical suture may be manufactured with a non-barbed lead at the leading end of the suture. Optionally, the single-ended barbed surgical suture may include a T-shaped member or other stop device on the trailing end of the suture. Optionally, the single-ended barbed surgical suture may be made with depth markings on the suture needle and/or on the suture body.

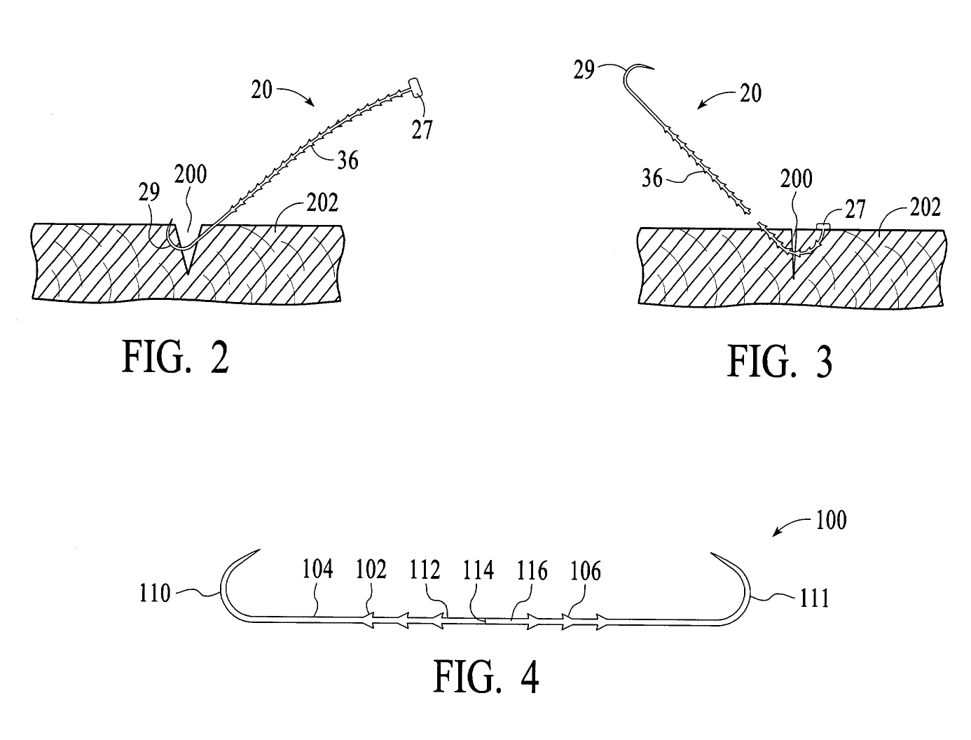

FIG. 2 shows the single-ended barbed surgical suture 20 of FIG. 1A being used to close a surgical incision 100. The edges of the incision 200 are pierced with the surgical needle 29 and the barbed surgical suture 20 is drawn through the tissue 202. The barbs 36 flex inward as they pass through the tissue 202, allowing the barbed surgical suture 20 to move easily through the tissue 202 in the forward direction. FIG. 3 shows the completed closure of the surgical incision 200 using the single-ended barbed surgical suture 20 of FIG. 1A. The closure is completed by drawing the barbed surgical suture 20 through the tissue 202 until the T-shaped member 27 on the trailing end of the suture 20 contacts the tissue 202 surface on the first side of the incision 200 while placing a small amount of pressure with a gloved finger or instrument on the tissue 202 surface on the second side of the incision 200 where the suture 20 exits. The barbs 36 expand or flex inward or outward to grip the tissue 202 and to resist movement in the reverse direction. Once good apposition is achieved, the leading end of the barbed surgical suture 20 can be cut off at the tissue 202 surface. Additional barbed surgical sutures 20 can be placed in this way along the incision 200 until a good closure is achieved. If desired, the barbed surgical sutures 20 can be placed at different depths to achieve proper apposition of multiple layers of tissue 202. Once the incision 200 is sufficiently healed, the barbed surgical suture 20 can be easily removed by cutting off the T-shaped member 27 on the trailing end of the suture 20 at the tissue 202 surface and pulling the barbed surgical suture 20 through the tissue 202 in the forward direction. The single-ended barbed surgical suture 20 may be manufactured with either a curved or straight suture needle 29 attached or detachably attached to the suture body 22.

The barbed surgical sutures 20, 30, 40, 50, 60, 67, 70, 80, 90, 100 place a sufficient amount of compression on the tissue 202 at the incision to promote healing without over compressing or distorting the tissue 202. When standard surgical sutures are tied, the tissue within the suturing line, especially near the knots, is usually over compressed, thereby hindering the healing process. Holding the tissue together without over compressing part of the tissue, as is possible with the barbed surgical suture of the present invention, promotes better wound healing. This feature allows the barbed surgical suture to achieve excellent cosmetic results with a minimum of scarring, superior even to standard knotted sutures. For this reason, the barbed surgical suture is very well suited for skin and tissue closures in cosmetic surgery and other surgeries where good cosmesis is important.

The barbed surgical suture 20, 30, 40, 50, 60, 67, 70, 80, 90, 100 may also be configured as a double-ended suture 100 with the barbs 102 on a first end portion 104 aligned to allow the suture 100 to move through tissue in a one direction and the barbs 106 on a second end portion 108 aligned to allow the suture 100 to move through tissue in the opposite direction. FIG. 4 shows a double-ended barbed surgical suture 100 constructed according to the present invention. The barbs 102, 106 on the double-ended barbed surgical suture 100 may be made in many different configurations, including those shown in FIGS. 1D, 1E, 1F, 1G, 7B and 8B. The double-ended barbed surgical suture 100 may be manufactured with curved or straight suture needles 110, 111 attached at one or both ends of the suture 100. The suture needles 110, 111 may be permanently attached or removably attached to the barbed surgical suture 100 or, alternatively, the suture needles 110, 111 may be integrally formed with the barbed surgical suture 100. Optionally, the double-ended barbed surgical suture 100 may be manufactured with a non-barbed lead at each end of the suture and/or with a non-barbed connecting member 112 between the two barbed portions 104, 108. Optionally, the double-ended barbed surgical suture 100 may be made with a mark 114 or other feature to indicate the center of the suture 100 between the two barbed portions 104, 108 and/or with depth markings on the suture needle 110, 111 and/or on the suture body 116. The suture body 116 may also be scored or weakened at a point between the two opposing barbed portions 104, 108. The weakened point would provide a joint where the suture body 116 could be broken or cut to make facilitate removal of the barbed surgical suture 100.

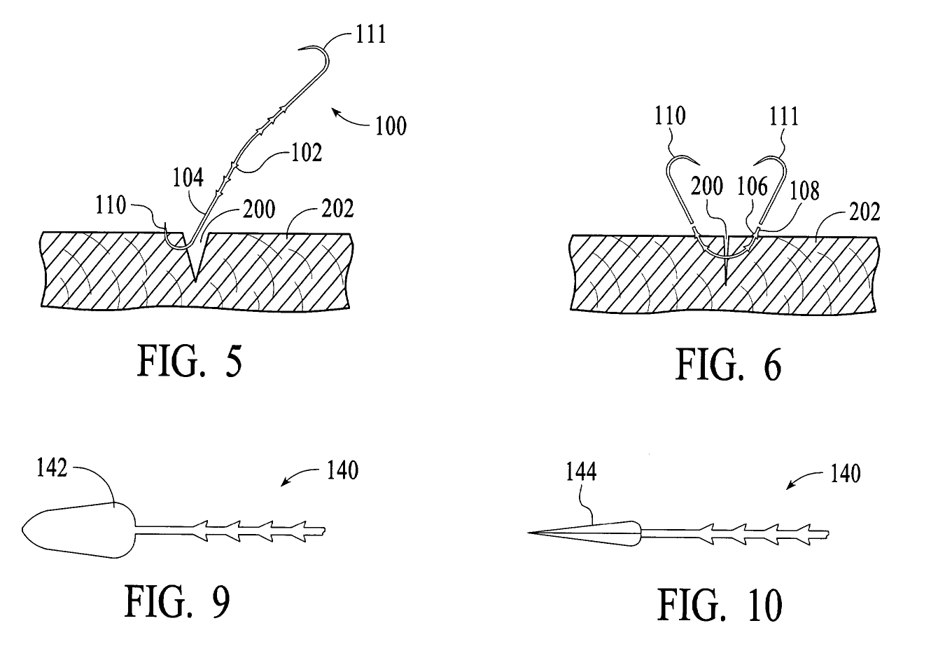

FIG. 5 shows the double-ended barbed surgical suture 100 of FIG. 4 being used to close a surgical incision 200. The incised edge of the tissue 202 on a first side of the incision 200 is pierced with a first surgical needle 110 on the first end of the double-ended barbed surgical suture 100 and the first barbed portion 104 is drawn through the tissue 202 until the center of the suture 100 is approximately at the incised edge of the tissue 202. The barbs 102 on the first barbed portion 104 flex inward as they pass through the tissue 202, allowing the first barbed portion 104 of the barbed surgical suture 100 to move easily through the tissue 202 in a forward direction. Then, the incised edge of the tissue 303 on the second side of the incision 200 is pierced with the second surgical needle 111 on the second end of the double-ended barbed surgical suture 100 and the second barbed portion 108 is drawn through the tissue 202 while placing a small amount of pressure with a gloved finger or instrument on the tissue 202 surface on the second side of the incision 200 where the suture 100 exits. The barbs 106 on the second barbed portion 108 flex inward as they pass through the tissue 202, allowing the second barbed portion 108 of the barbed surgical suture 100 to move easily through the tissue 202 in a forward direction, while the barbs 102 on the first barbed portion 104 expand or flex inward or outward, depending on the barb configuration, to grip the tissue 202 and to resist movement in the reverse direction. Once good apposition is achieved, both ends of the double-ended barbed surgical suture 100 can be cut off at the tissue 202 surface. FIG. 6 shows the completed closure of the surgical incision 200 using the double-ended barbed surgical suture 100 of FIG. 4. Additional barbed surgical sutures 100 can be placed in this way along the incision 200 until a good closure is achieved. If desired, the barbed surgical sutures 100 can be placed at different depths to achieve proper apposition of multiple layers of tissue 202.

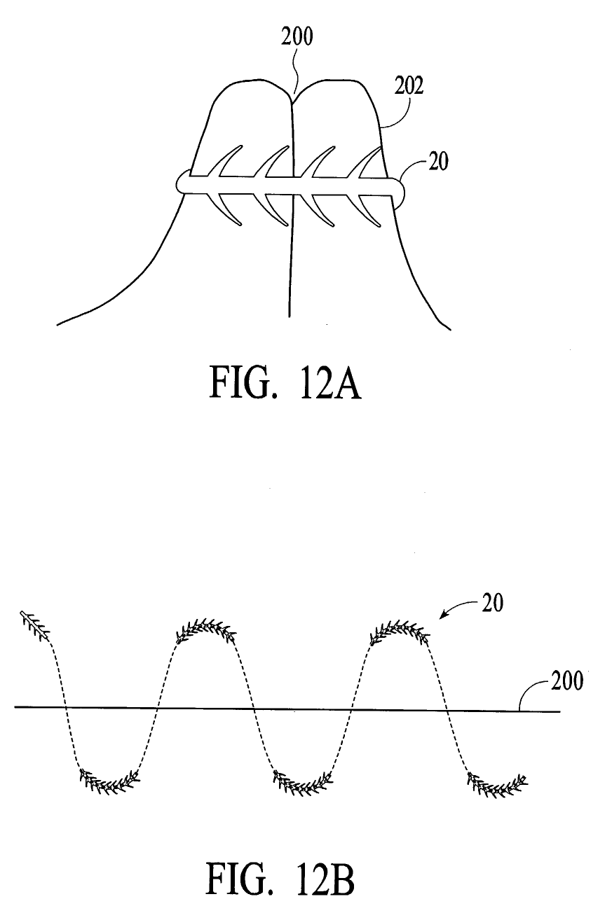

An alternate method of using the barbed surgical suture 20, 30, 40, 50, 60, 67, 70, 80, 90, 100 would use multiple passes of a single suture to form a continuous stitch that zigzags back and forth through the wound or incision. FIG. 12A shows side view of a wound closure and FIG. 12B shows a top view thereof. This procedure would be performed using one or more long barbed surgical sutures. With a single-ended suture, as seen in FIGS. 1A, 7A, and 8A, the wound closure could begin at one end of the wound and work towards the other end. If a greater length is needed, additional barbed surgical sutures could be used to finish closing the wound. The first passes through the tissue might also be made near the center of the wound using a first barbed surgical suture and worked toward one end. A second suture would be used to work towards the other end of the wound. If a double-ended suture is used, such as shown in FIGS. 4-6, the first passes through the tissue could begin at the middle of the wound and work toward one end. The other end of the barbed surgical suture could then be worked towards the other end. Alternately, the multiple sutures or multiple passes with a single suture may be made to reinforce the closure if necessary, such as where the integrity of the tissue is in question. The multiple passes may be made at a fairly consistent depth in the wound, or the multiple passes may be used at different depth to hold the tissue together at different layers.

The barbed surgical suture 20, 30, 40, 50, 60, 67, 70, 80, 90, 100 may also be used to quickly close wounds in trauma patients. Field technicians could use the barbed surgical suture to quickly close a wound to reduce bleeding during transport of a patient to a location capable of providing the level of care required. Situations where this treatment could be appropriate range from stopping bleeding of battle wounds during transport to a safe location for treatment to stopping or reducing bleeding during extrication of a crash injury victim to blood loss reduction during ambulance transport to an emergency room.

The configuration of the barbed surgical suture 20, 30, 40, 50, 60, 67, 70, 80, 90, 100 lends itself to economical methods of manufacture. In a first exemplary method, the barbed surgical suture can be manufactured by injection molding. The mold can be produced using an EDM process by first machining an EDM electrode in the shape and size of the barbed suture in carbon or other suitable material. The EDM electrode is then used to burn a mold cavity in the shape and size of the barbed suture into the mold. The EDM electrode may include the shape of the runners and gates as well or these features can be added using conventional machining. Preferably, the barbed suture is injection molded on a high-pressure injection molding press using a low melt viscosity molding resin to facilitate filling the mold cavity. The use of multiple mold gates spaced along the suture body and/or a heated mold may also help to facilitate mold filling.

Polymeric materials suitable for injection molding the barbed surgical suture 20, 30, 40, 50, 60, 67, 70, 80, 90, 100 include, but are not limited to: polyethylene, polypropylene, polyimide, polyamide (e.g. Nylon 6 and Nylon 66), polyester and polycarbonate. Alternatively, bioabsorbable materials, such as polylactic acid, polyglycolic acid, polyglactin, polyepsilon-caprolactone, polydioxanone, polyortho ester, polyethylene oxide, and/or their copolymers can be used for injection molding the barbed surgical suture.

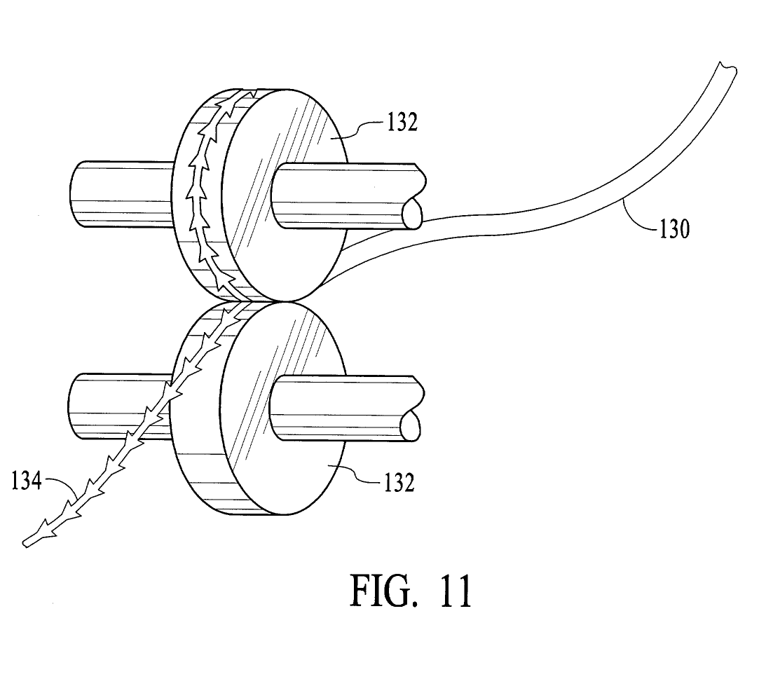

In a second exemplary method, the barbed surgical suture 20, 30, 40, 50, 60, 67, 70, 80, 90, 100 can be manufactured by stamping or cutting the suture out of a flat material. The flat material may be extruded, rolled or cast into a flat ribbon or sheet. A cutting die in the shape of the barbed surgical suture may be used to cut the barbed surgical suture out of the flat material. FIG. 11 illustrates a manufacturing process for producing a barbed surgical suture from a flat material 130 using a rolling cutting die 132 or a progressive die. The rolling cutting die 132 may be configured to cut out a continuous length of barbed surgical suture 134 or it may be configured to cut out a complete barbed surgical suture in a discrete length with all of the product features, including for example the optional T-shaped member and/or one or more integrally formed suture needles. The exemplary rolling cutting die or progressive die shown in FIG. 11 is configured to cut out a single barbed surgical suture from a narrow ribbon-shaped flat material. In alternate embodiments, the rolling cutting die or progressive die may be configured to simultaneously cut out several barbed surgical sutures from a broad sheet of flat material. Alternatively, conventional machine tools, EDM, lasers, water jets or other cutting means may be used for cutting the suture out of flat material. However, these methods may not be as well suited for economical high volume production.

Materials suitable for manufacturing the barbed surgical suture 20, 30, 40, 50, 60, 67, 70, 80, 90, 100 include, but are not limited to: polyethylene, polypropylene, polyimide, polyamide (e.g. Nylon 6 and Nylon 66), polyester and polycarbonate. Alternatively, bioabsorbable materials, such as polylactic acid, polyglycolic acid, polyglactin, polyepsilon-caprolactone, polydioxanone, polyortho ester, polyethylene oxide, and/or their copolymers can be used for injection molding the barbed surgical suture. This manufacturing method can also be used for producing barbed surgical sutures from thin metal sheets.

In a third exemplary method, the barbed surgical suture 20, 30, 40, 50, 60, 67, 70, 80, 90, 100 can be manufactured by chemically etching the suture out of flat material. In one particularly preferred variation of this method, the barbed surgical suture is etched from a flat sheet of metal using a photoetching or photochemical etching process. The photoetching process can be used to produce long lengths and or any desired features to be incorporated of barbed surgical suture arranged on a sheet of material or it may be used to produce a complete barbed surgical suture in a discrete length with all of the product features, including for example the optional T-shaped member and/or one or more integrally formed suture needles.

Materials suitable for manufacturing the barbed surgical suture 20, 30, 40, 50, 60, 67, 70, 80, 90, 100 by this method include, but are not limited to: stainless steel (e.g. 302, 304 and 316 series stainless steel), cobalt-iron alloys (e.g. Elgiloy and Carpenter MP35), nickel and nickel alloys (e.g. alloy 42), and nickel-titanium alloys.

A curved or straight suture needle may be integrally formed with the barbed surgical suture 20, 30, 40, 50, 60, 67, 70, 80, 90, 100. FIGS. 9-10 illustrate two steps of a manufacturing process for producing a barbed surgical suture 140 with an integrally formed suture needle 144. Using a photoetching, cutting, stamping or progressive die process as described above, the barbed surgical suture 140 is produced with a needle blank 142 attached at one or both ends of the barbed surgical suture 140, as shown in FIG. 9. Then, the needle blank 142 is formed into a curved or straight suture needle 144 while it is still attached to the barbed surgical suture 140, as shown in FIG. 10. The needle blank 142 may be rolled, folded, swaged and/or crimped to form the suture needle 144. If desired, the end of the needle 144 may subsequently be sharpened, for example by grinding. Alternately, the barbed surgical suture may include an integrally molded polymer needle or have a molded or shaped polymer needle attachable thereto.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof.

What is claimed is:

1. A surgical suture comprising:
a flexible elongated suture body, said elongated suture body being substantially flat and having a first lateral edge and a second lateral edge; and
a multiplicity of barbs located along said first lateral edge of said elongated suture body.

2. The surgical suture of claim 1, further comprising a second multiplicity of barbs located along a second lateral edge of said elongated suture body.

3. The surgical suture of claim 1, wherein said elongated suture body has a first end and a second end and wherein said multiplicity of barbs are configured to allow said elongated suture body to move through tissue in the direction of said first end and to resist said elongated suture body moving through tissue in the direction of said second end.

4. The surgical suture of claim 3, further comprising a surgical needle attached to said first end of said elongated suture body.

5. The surgical suture of claim 4, further comprising an approximately T-shaped member attached to said second end of said elongated suture body.

6. The surgical suture of claim 3, further comprising a surgical needle removably attached to said first end of said elongated suture body.

7. The surgical suture of claim 6, wherein said elongated suture body, said barbs and said surgical needle are integrally formed of a metal.

8. The surgical suture of claim 6, wherein said elongated suture body, said barbs and said surgical needle are integrally formed of a polymer.

9. The surgical suture of claim 6, wherein said elongated suture body, said barbs and said needle are formed of a bioabsorbable material.

10. The surgical suture of claim 3, further comprising a T-shaped member attached to said second end of said elongated suture body.

11. The surgical suture of claim 3, further comprising a surgical needle connected to said first end and integrally formed with said elongated suture body.

12. The surgical suture of claim 1, wherein said elongated suture body and said barbs are formed of a polymer.

13. The surgical suture of claim 1, wherein said elongated suture body and said barbs are integrally molded of a polymer.

14. The surgical suture of claim 1, wherein said elongated suture body and said barbs are formed of a metal.

15. The surgical suture of claim 1, wherein said elongated suture body and said barbs are integrally formed of a metal.

16. The surgical suture of claim 1, wherein said elongated suture body and said barbs are formed by photoetching a metal.

17. The surgical suture of claim 1, wherein said elongated suture body and said barbs are formed of a bioabsorbable material.

18. The surgical suture of claim 1, further comprising a coating covering said suture body and said barbs.

19. The surgical suture of claim 1, further comprising a coating of silicone covering said suture body and said barbs.

20. The surgical suture of claim 1, further comprising a coating of TEFLON covering said suture body and said barbs.

21. The surgical suture of claim 1, further comprising a plated coating of covering said suture body and said barbs.

22. The surgical suture of claim 1, wherein said elongated suture body has a first end with a first end portion proximate said first end and a second end with a second end portion proximate said second end, wherein the barbs on said first end portion of said elongated suture body are configured to allow said first end portion to move through tissue in the direction of said first end and to resist said first end portion moving through tissue in the direction of said second end, and wherein the barbs on second first end portion of said elongated suture body are configured to allow said second end portion to move through tissue in the direction of said second end and to resist said second end portion moving through tissue in the direction of said first end.

23. The surgical suture of claim 22, further comprising a marking on said suture body, said marking located between said first end portion and said second end portion.

24. The surgical suture of claim 22, further comprising a weakened location on said suture body, said weakened located between said first end portion and said second end portion.

25. The surgical suture of claim 22, further comprising a first surgical needle attached to said first end of said elongated suture body and a second surgical needle attached to said second end of said elongated suture body.

26. The surgical suture of claim 25, wherein said elongated suture body, said barbs, said first surgical needle and said second surgical needle are integrally formed of a metal.

27. The surgical suture of claim 22, further comprising a first surgical needle removably attached to said first end of said elongated suture body and a second surgical needle removably attached to said second end of said elongated suture body.
